(19) United States
(12) Patent Application Publication
(10) Pub. No.: US 2017/0281327 A1
(43) Pub. Date: Oct. 5, 2017

(54) SLEEVE FOR SOFT TISSUE REPAIR

(71) Applicants: Lee D. Kaplan, Coconut Grove, FL (US); James P. Bradley, Pittsburgh, PA (US)

(72) Inventors: Lee D. Kaplan, Coconut Grove, FL (US); James P. Bradley, Pittsburgh, PA (US)

(21) Appl. No.: 15/476,128

(22) Filed: Mar. 31, 2017

Related U.S. Application Data
(60) Provisional application No. 62/316,723, filed on Apr. 1, 2016.

(51) Int. Cl. A61F 2/00; A61B 17/06

(52) U.S. Cl. CPC A61F 2/0063; A61B 17/06166; A61B 2017/00884

(57) ABSTRACT

A sleeve for soft tissue repair includes a cylindrical tube formed of at least one biocompatible material, and a suture tethered to one end of the cylindrical tube. The cylindrical tube is configured to be fitted over a soft tissue. The cylindrical tube is configured to reversibly and repeatedly deform from a relaxed state to a stretched state in which the cylindrical tube contracts in a radial direction and expands in a longitudinal direction to secure the soft tissue within the cylindrical tube.

### CROSS-REFERENCE TO RELATED APPLICATIONS

This application claims the benefit of U.S. Provisional Application No. 62/316,723 filed on Apr. 1, 2016, which is hereby incorporated by reference in its entirety.

### FIELD

The present disclosure generally related to a device for soft tissue repair. More specifically, the disclosure relates to a sleeve and method for securing soft tissue during soft tissue repair.

### BACKGROUND

Soft tissue includes tendons, ligaments, fascia, skin, fibrous tissues, fat, and synovial membranes (which are connective tissue), and muscles, nerves and blood vessels (which are not connective tissue). When soft tissue is damaged, the damage may be in the form of a partial tear in which the soft tissue is not completely severed into two pieces, or complete tears in which the soft tissue is severed into two pieces. In many cases, tears begin with fraying of the tissue.

A need exists for improved technology, including technology related to medical devices for stabilizing soft tissue to prevent frayed or partially torn tissue from becoming fully torn, and to assist in the healing process after undergoing a surgical procedure to repair soft tissue.

### SUMMARY

In a first aspect, a sleeve for soft tissue repair includes a cylindrical tube formed of at least one biocompatible material, and a suture tethered to one end of the cylindrical tube. The cylindrical tube is configured to be fitted over a soft tissue. The cylindrical tube is configured to reversibly and repeatedly deform from a relaxed state to a stretched state in which the cylindrical tube contracts in a radial direction and expands in a longitudinal direction to secure the soft tissue within the cylindrical tube. The suture may be a standard thread-like suture or an asymmetric suture.

In another aspect, a system of soft tissue repair includes a sleeve comprising a cylindrical tube formed of at least one biocompatible material, a suture tethered to one end of the cylindrical tube, and at least one suture anchor configured to secure the suture to the soft tissue, a muscle, or a bone. The cylindrical tube is configured to be fitted over a soft tissue. The cylindrical tube is configured to reversibly and repeatedly deform from a relaxed state to a stretched state in which the cylindrical tube contracts in a radial direction and expands in a longitudinal direction to secure the soft tissue within the cylindrical tube. The suture may be a standard thread-like suture or an asymmetric suture.

In yet another aspect, method for soft tissue repair includes providing a sleeve over a soft tissue to be repaired, the sleeve comprising a cylindrical tube formed of at least one biocompatible material, and a suture tethered to one end of the cylindrical tube; pulling the suture to deform the cylindrical tube from a relaxed state to a stretched state in which the cylindrical tube contracts in a radial direction and expands in a longitudinal direction to secure the soft tissue within the cylindrical tube; and securing the suture to maintain a radial force imparted to the soft tissue by the sleeve in the stretched state. The suture may be a standard thread-like suture or an asymmetric suture. The suture may be secured using at least one suture anchor configured to secure the suture to the soft tissue, a muscle, or a bone

### BRIEF DESCRIPTION OF THE DRAWINGS

FIG. 1 is an illustration of the anatomy of a muscle connected to a soft tissue.

FIG. 2 is an illustration of a first embodiment of a sleeve disposed around the soft tissue of FIG. 1 in a relaxed state.

FIG. 3 is an illustration of the sleeve of FIG. 2 disposed around the soft tissue in a stretched state.

FIG. 4 is an illustration of a second embodiment of a sleeve disposed around the soft tissue of FIG. 1 in a relaxed state.

FIG. 5 is an illustration of the sleeve of FIG. 4 disposed around the soft tissue in a stretched state.

FIG. 6 is an illustration of a first method of securing a suture attached to a distal end of the sleeve of FIG. 3 or FIG. 5.

FIG. 7 is an illustration of a second method of securing a suture attached to a distal end of the sleeve of FIG. 3 or FIG. 5.

FIG. 8 is an illustration of a third method of securing a suture attached to a distal end of the sleeve of FIG. 3 or FIG. 5.

FIG. 9 is an illustration of a fourth method of securing a suture attached to a distal end of the sleeve of FIG. 3 or FIG. 5.

FIG. 10 is an illustration of a fifth method of securing a suture attached to a distal end of the sleeve of FIG. 3 or FIG. 5.

FIG. 11 is an illustration of examples of braided patterns that may be used to form the sleeve of FIGS. 2-5.

### DESCRIPTION

As used herein, the term “double-row” refers to a suture configuration including a medial row and a lateral row. The term “medial row” refers to the suture medially passing through the soft tissue from a top surface of the soft tissue (or sleeve) to a bottom surface (i.e., a surface closest to a bone) of the soft tissue (or sleeve) or vice versa. The term “lateral row” refers to the suture laying across the top surface of the soft tissue (or sleeve) from a first point on the top surface to a second point on the top surface of the soft tissue (or sleeve).

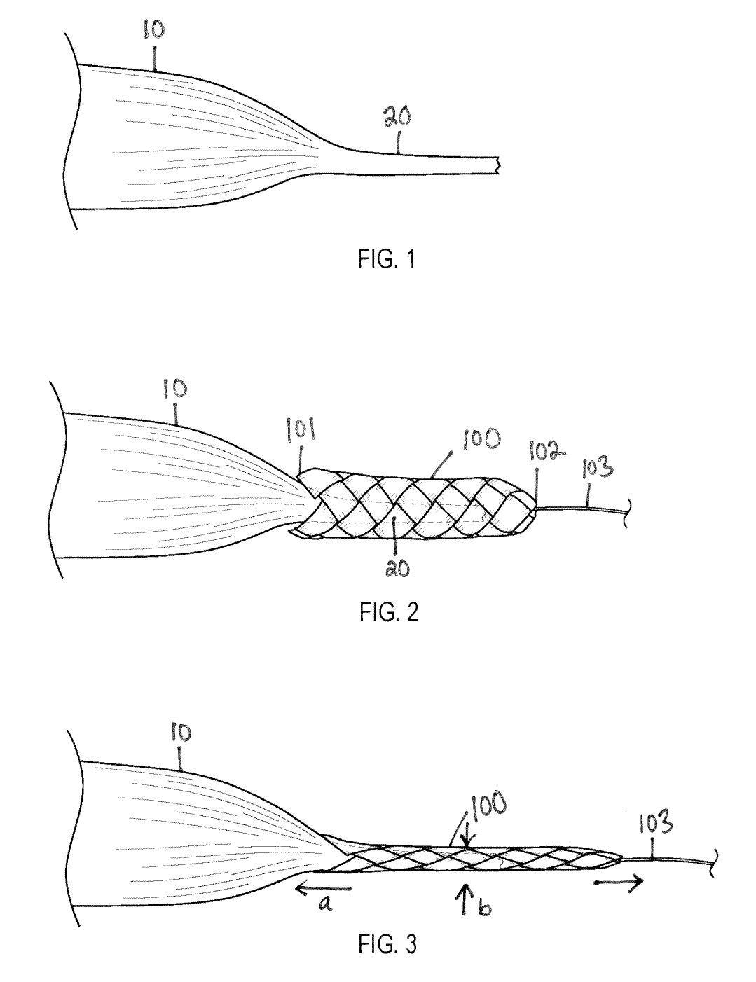

Referring to FIG. 1, a muscle 10 is connected to a soft tissue 20. The soft tissue 20 attaches the muscle 10 to a bone (not illustrated). In one example, the soft tissue 20 is a biceps tendon that attaches a biceps muscle 10 to bones in the shoulder and in the elbow. In other examples, the soft tissue 20 may be the anterior cruciate ligament (ACL), hand tendon, Achilles tendon, patella tendon, quadriceps tendon, etc. One of ordinary skill in the art will understand that the sleeve 100 may be used in the repair of any soft tissue.

Referring to FIGS. 2-5, in one embodiment, a hollow, sleeve 100 is placed around a circumference of the soft tissue 20. The sleeve 100 is a cylindrical tube having an open, proximal end 101 and an open, distal end 102. The proximal end 101 is the end closest to the muscle 10. The sleeve 100 is made from a lattice structure configured to contract in a radial direction (“b” arrows in FIGS. 3 and 5) when expanded in a longitudinal direction (“a” arrows in FIGS. 3 and 5). Prior to use, the sleeve 100 may be provided on a loading structure (e.g., a cylindrical structure such as a straw or a funnel-shaped structure) and rolled or slid onto the soft tissue 20. To facilitate transfer of the sleeve 100 from the loading structure to the soft tissue 20, the sleeve 100 may have an internal diameter that is larger than an external diameter of both the loading structure and the soft tissue 20. For example, the sleeve 100 may have an internal diameter of 7-10 mm, while the loading structure and the soft tissue 20 may have an external diameter of 6 mm or less. These dimensions are illustrative only, and one of ordinary skill in the art would appreciate that the dimensions of the sleeve 100, the loading structure, and the soft tissue 20 are not limited to these ranges.

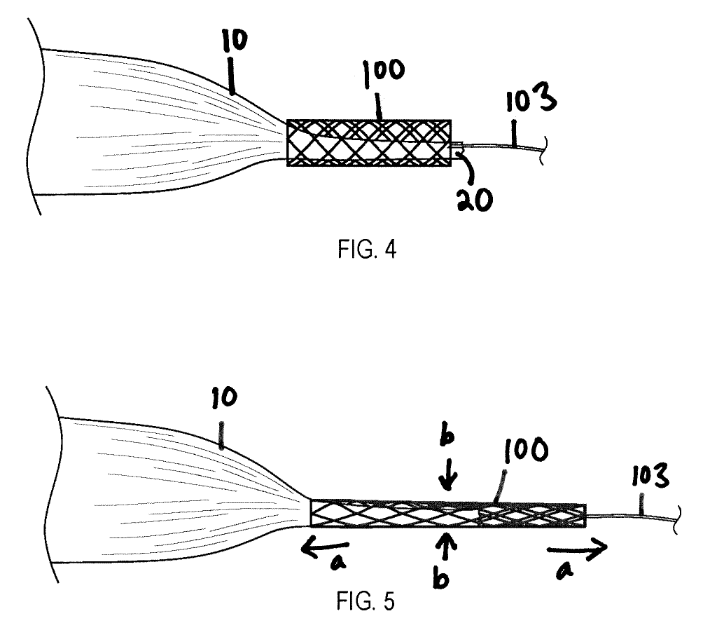

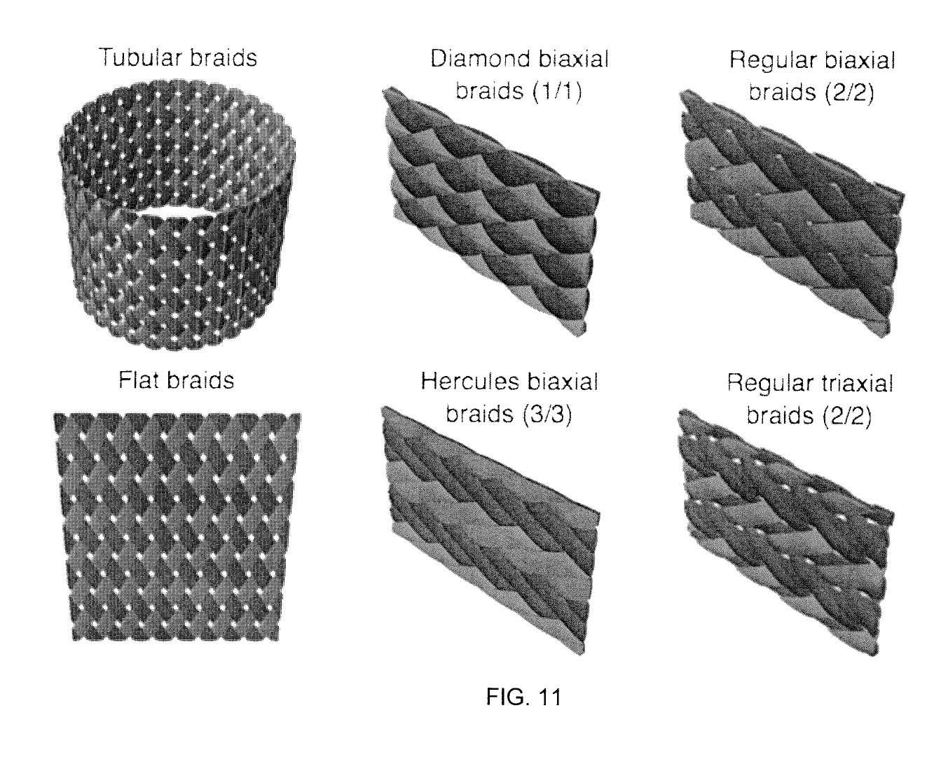

In the embodiment of FIGS. 2 and 3, the sleeve 100 is comprised of a helically wound braided material. In particular, strips of a biocompatible materials are braided together to form the sleeve 100. The braid may be, for example, a biaxial braid (e.g., a diamond biaxial braid, a regular biaxial braid or a Hercules biaxial braid) or a triaxial braid. Examples of braided patterns are illustrated in FIG. 11. In the embodiment of FIGS. 4 and 5, the sleeve 100 is comprised of a mesh material formed by interlacing wires or fibers of biocompatible materials together. The wires or fibers may be interlaced in patterns similar to the braided patterns of FIG. 11, with the wires or fibers occupying areas corresponding to the edges of the strips seen in FIGS. 2 and 3. Spaces are provided between adjacent wires or fibers. The biocompatible material may be any biocompatible material capable of expanding in a longitudinal direction and contracting in a radial direction when formed into a cylindrical tube. For example, the biocompatible material may be suture material, tissue graft, plastic, polymers or any combination thereof. The biocompatible material is arranged such that the cylindrical tube is continuous along an entire length thereof. Edges of the biocompatible material do not need to be tied together to form or maintain a shape of the cylindrical tube.

In certain embodiments, the sleeve 100 is impregnated with biologic agent or used in conjunction with a biologic agent, where the biologic agent may assist with healing of tissue. The biologic may be provided in interstitial spaces between the strips, wires or fibers that comprise the sleeve 100. The biologic may be a sugar, a protein, a nucleic acid, a cell, tissue, or complex combinations of any two or more such substances. The biologic agent may include, but is not limited to, stem cells, growth factors, hormones, blood, tissues used for transplantation (e.g., tendons, ligaments and bone), and the like or mixtures of any two or more such agents. In some embodiments, the sleeve 100 may be a scaffold incorporating growth factors or stem cells for tissue or bone engineering.

A suture or suture tape 103 (hereinafter “suture 103”) is tethered to the distal end 102 of the sleeve 100. The suture 103 may be a standard suture or an asymmetrical suture, as described in U.S. Pat. No. 9,144,425, the entire contents of which are incorporated herein by reference in their entirety for disclosures related to structures of asymmetrical sutures. In embodiments including asymmetrical sutures that are asymmetric in the sense that different regions along a length of the suture have different widths, it is preferable that the region having the greatest width forms a lateral row on an exterior surface of the sleeve 100 to provide a greater contact area between the asymmetric suture and the sleeve 100, and to even a distribution of pressure on the sleeve 100. In other examples (not illustrated), a plurality of sutures 103 may be tethered to the distal end 102 of the sleeve 100, one or more sutures 103 may be tethered to each of the proximal end 101 and the distal end 102 of the sleeve 100, or one or more sutures 103 may be tethered to only the proximal end 101 of the sleeve 100 (i.e., no sutures 103 tethered to the distal end 102 of the sleeve 100).

The sleeve 100 may be provided with the suture 103 already attached, or the suture 103 may be attached by the surgeon. When the surgeon pulls on the suture 103, the sleeve 100 contracts in a radial direction (“b” arrows in FIGS. 3 and 5) and expands in a longitudinal direction (“a” arrows in FIGS. 3 and 5), thereby securing the soft tissue 20 via the radial force imparted on the soft tissue 20 by the sleeve 100.

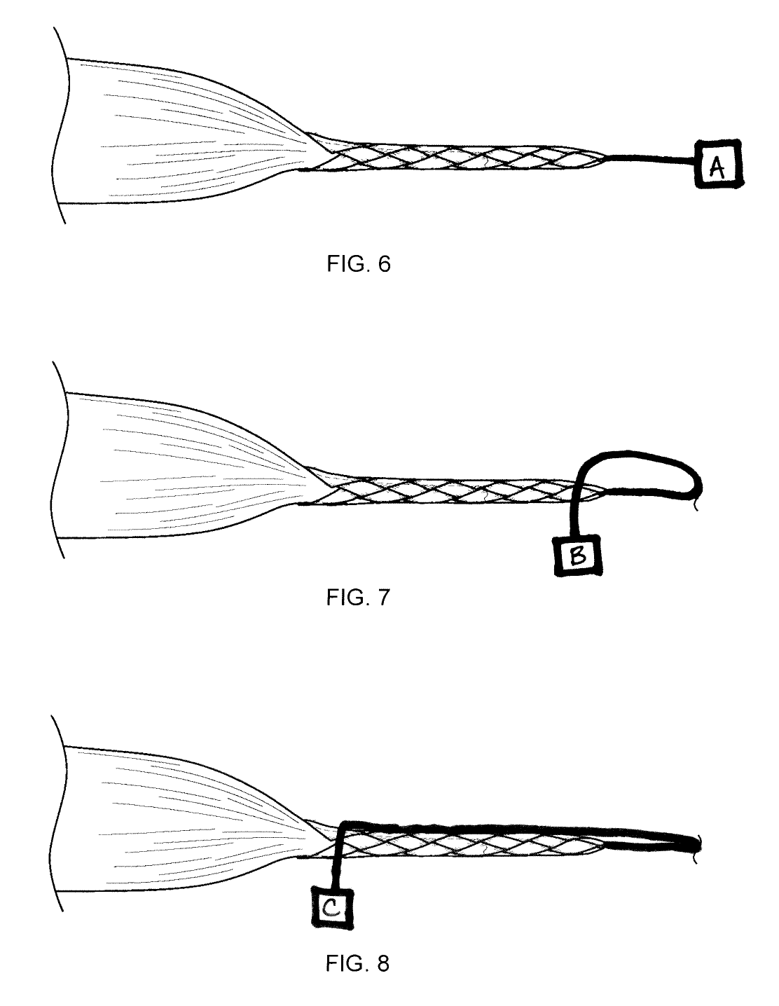

Referring to FIGS. 6-10, the suture 103 can be secured in one or more positions A, B, C or D. Although the embodiment of FIGS. 2 and 3 is illustrated in FIGS. 6-10, one of ordinary skill in the art would understand that the positions and methods for securing the suture described with respect to FIGS. 6-10 may also be used to secure the suture in the embodiment of FIGS. 4 and 5.

Referring to FIGS. 6-10, position A is located distal to the sleeve 100 and may be within, for example, a neighboring muscle, tissue or bone. Position B is located beneath the distal end 102 of the sleeve 100 and may be within, for example, an underlying muscle, tissue or bone. Position C is located beneath the proximal end 101 of the sleeve 100 and may be within, for example, an underlying muscle, tissue or bone. Position D is located proximal to the sleeve 100 and may be within, for example, the muscle 10.

In the embodiments in which the suture 103 comprises suture tape, the distal end of the suture tape may be adhered in any one of positions A or D.

In the embodiments in which the suture 103 comprises a thread-like suture, the suture 103 may be locked directly into the muscle, tissue or bone, or secured by a suture anchor or suture button (e.g., a two-hole suture button or a four-hole suture button) at one or more of the positions A, B, C or D. In the embodiments of FIGS. 8-10, a lateral row is formed across the top surface of the sleeve 100. Moreover, in order to ensure that the sleeve 100 is secured to the soft tissue 20, the suture may be passed through the sleeve 100, or through the sleeve 100 and the soft tissue 20, in between one or more of the positions A-D to form medial rows and lateral rows configured to secure the sleeve 100 to the soft tissue 20.

Depending on the type of suture anchor used, the suture 103 may be “knotless” in that it is unnecessary to tie a knot in the suture 103 to secure a distal end of the suture 103. In certain embodiments including a knotless suture, a suture anchor comprised of an anchor body configured to receive a plug to secure the suture 103 between the distal end of the plug and an inner bottom surface of the anchor body may be placed in one or more of positions A, B, C or D. Examples of such a suture anchor can be found in U.S. Patent Application Publication No. 2008/0077161, U.S. Patent Application Publication No. 2011/0009884, U.S. Pat. No. 8,202,295, and U.S. Pat. No. 9,144,425, the entire contents of which are incorporated herein by reference in their entireties for their disclosures related to structures of suture anchors. Each of these suture anchors allows for tightening, adjustment, or re-tensioning of a suture by tightening, loosening, re-tightening, and/or removing the plug from the anchor body. The suture anchors also allow for securing of the suture 103 without the tying of knots or replacement of the suture 103 when re-tensioning is required.

In certain embodiments, a first suture anchor may be placed in position B and a second suture anchor may be placed in position C (see FIG. 10). One or both of the first suture anchor and the second suture anchor may be a suture anchor comprised of an anchor body configured to receive a plug to secure the suture 103 between the distal end of the plug and an inner bottom surface of the anchor body, as described in U.S. Patent Application Publication No. 2008/0077161, U.S. Patent Application Publication No. 2011/0009884, U.S. Pat. No. 8,202,295, or U.S. Pat. No. 9,144,425.

Alternatively, the first suture anchor may be a pulley anchor configured to allow the tension of the suture 103 to be altered without locking the suture 103. An example of such a pulley anchor is described in U.S. Pat. No. 9,144,425, the entire contents of which is incorporated herein by reference in their entirety for disclosures related to structures of a pulley anchor. In this embodiment, the second suture anchor may be a suture anchor comprised of an anchor body configured to receive a plug to secure the suture 103 between the distal end of the plug and an inner bottom surface of the anchor body, as described in U.S. Patent Application Publication No. 2008/0077161, U.S. Patent Application Publication No. 2011/0009884, U.S. Pat. No. 8,202,295, or U.S. Pat. No. 9,144,425. The configuration illustrated in FIG. 10, is a “double-row” configuration including a lateral row (e.g., across the top surface of the sleeve 100) and a medial row (e.g., from the top surface of the distal end 102 of the sleeve 100 to the bottom surface of the distal end of the sleeve 100).

In some embodiments, the suture 103 is locked or prevented from being pulled back in a second direction after the suture 103 is threaded in a suture anchor or suture button in a first direction. In these embodiments, the suture anchor may be a check valve that allows the suture 103 to be threaded in a first direction through the suture anchor, but does not allow the suture 103 to be pulled back in a second direction, opposite to the first direction.

In other embodiments, the suture 103 is a barbed suture including arrow heads, or half-arrow heads such as the barb on a fishing hook, disposed at predetermined intervals along the length of the suture 103. Similar to the check valve, the arrow heads allow the suture 103 to be threaded in a first direction through the suture anchor, but the arrow heads do not allow the suture 103 to be pulled backing a second direction, opposite to the first direction.

The suture anchors described above may be made from a variety of materials known to those of skill in the art. For example, the material used for the suture anchor is typically a rigid biocompatible material such as a metal, a polymer, or a ceramic. Biocompatible metals include, but are not limited to stainless steel, titanium, tantalum, aluminum, chromium, molybdenum, cobalt, silver, and gold, or alloys of such metals that are known to those of skill in the art. Biocompatible polymers include, but are not limited to, high-density polyethylenes, polyurethanes, or blends of such polymers, as are known to those of skill in the art. Biocompatible polymers also include absorbable materials such as polylactic acid, polyglycolic acid, or mixtures thereof. Biocompatible ceramics include, but are not limited to alumina, silica, silicon carbide, silicon nitride, zirconia, and mixtures of any two or more thereof.

For suture anchors including plugs, the plugs may likewise be prepared from similar metals, polymers, and ceramics. However in some embodiments, the anchor plugs are prepared from materials that may be compressed. In such embodiments, the plug material is capable of being compressed from an uncompressed state to a compressed state, prior to or during insertion of the plug into the anchor body. Such compression allows for the material to recoil from the compressed state to the uncompressed state and thereby increasing the friction fit between the plug and the anchor body. Such materials that may be compressed include, but are not limited to, polyethylenes, silicones, polyesters, polyurethanes, polylactic acid, polyglycolic acid, or mixtures of any two or more thereof.

Methods of using the sleeve 100 for soft tissue repair are also provided. The sleeve 100 is placed over the soft tissue 20 in a relaxed state (see FIGS. 2 and 4) such that the soft tissue is loosely encased by the sleeve 100. In the relaxed state of the sleeve 100, a gap may exist between the interior of the sleeve 100 and the exterior of the soft tissue 20. The suture 103 is tethered to the distal end 102 of the sleeve 100. The suture 103 is then pulled such that the sleeve 100 deforms to a stretched state in which the sleeve 100 contracts in a radial direction (“b” arrows in FIGS. 3 and 5) and expands in a longitudinal direction (“a” arrows in FIGS. 3 and 5), thereby securing the soft tissue 20 within the sleeve 100. In the stretched state of the sleeve 100, the interior surface of the sleeve 100 contacts the exterior surface of the soft tissue 20. The interior surface of the sleeve 100 may simply abut the exterior surface of the soft tissue 20, or the interior surface of the sleeve 100 may impart a weak radial force (i.e., a compressive force) onto the soft tissue 20. The sleeve 100 provides a larger contact area, and therefore, a better grasp of the soft tissue 20 than a point specific suture 103 alone. The sleeve 100 holds the soft tissue 20 in place to reinforce the soft tissue 20 and prevent further damage to the soft tissue 20. Moreover, the sleeve 100 may even a distribution of pressure on the soft tissue 20

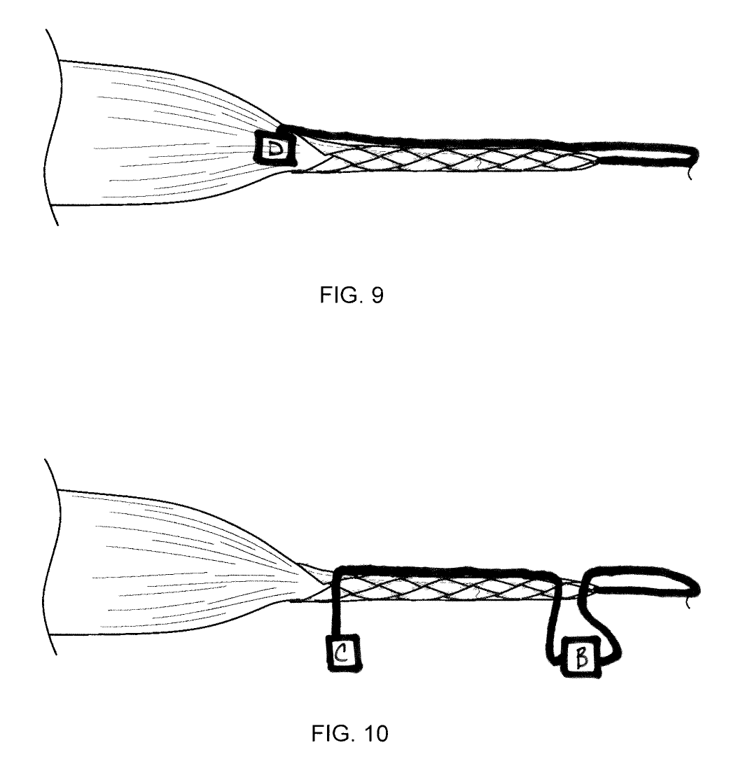

The distal end of the suture 103 is secured in neighboring or underlying muscle, tissue or bone by adhering or locking the suture 103 in a suture button or suture anchor. In FIG. 6, the suture 103 is secured at the position A, which is located distal to the sleeve 100 and within, for example, a neighboring muscle, tissue or bone. In FIG. 7, the suture 103 is secured at the position B, which is located beneath the distal end 102 of the sleeve 100 and within, for example, an underlying muscle, tissue or bone. To arrive at the position B, the suture 103 is looped to the top surface of the sleeve 100 at the distal end 102, inserted into the top surface of the sleeve 100, pulled through the soft tissue 20 and pulled out of the bottom surface of the sleeve 100. In FIG. 8, the suture 103 is secured at the position C, which is located beneath the proximal end 101 of the sleeve 100 and within, for example, an underlying muscle, tissue or bone. To arrive at the position C, the suture 103 is looped to the top surface of the sleeve 100 at the distal end 102, extended along the top surface of the sleeve 100 from the distal end 102 to the proximal end 101, inserted into the top surface of the sleeve 100 at the proximal end 101, pulled through the soft tissue 20 and pulled out of the bottom surface of the sleeve 100. In FIG. 9, the suture 103 is secured at the position D, which is located proximal to the sleeve 100 and within the muscle 10. To arrive at the position D, the suture 103 is looped to the top surface of the sleeve 100 at the distal end 102, extended along the top surface of the sleeve 100 from the distal end 102 to the proximal end 101. In FIG. 10, the suture 103 is secured at both positions B and C. To arrive at the position B, the suture 103 is looped to the top surface of the sleeve 100 at the distal end 102, inserted into the top surface of the sleeve 100, pulled through the soft tissue 20 and pulled out of the bottom surface of the sleeve 100. The suture 103 is looped through the suture anchor at position B, inserted into the bottom surface of the sleeve 100, pulled through the soft tissue 20 and pulled out of the top surface of the sleeve 100 at the distal end 102. The suture 103 is then extended along the top surface of the sleeve 100 from the distal end 102 to the proximal end 101, inserted into the top surface of the sleeve 100 at the proximal end 101, pulled through the soft tissue 20 and pulled out of the bottom surface of the sleeve 100.

In the embodiments in which a suture anchor is used that allows for re-tensioning of the suture 103, the contraction of the sleeve 100 in a radial direction may be adjusted by re-tensioning the suture 103. The sleeve 100 may be reversibly and repeatedly deformed from the relaxed state to the stretched state.

One of ordinary skill in the art would appreciate that a plurality of sutures 103 may be used in conjunction with the sleeve 100. For example, the plurality of sutures may be secured in a single suture button or suture anchor, or each of the sutures may be secured in one of a plurality of suture buttons or suture anchors.

For the purposes of this disclosure and unless otherwise specified, “a” or “an” means “one or more.”

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified. As will be understood, wherever the term “comprising” appears in the claims, it may be replaced in some embodiments with the term “consisting essentially of,” or “consisting of.”

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

What is claimed is:

1. A sleeve for soft tissue repair comprising:
a cylindrical tube formed of at least one biocompatible material; and
a suture tethered to one end of the cylindrical tube,
wherein:
the cylindrical tube is configured to be fitted over a soft tissue, and
the cylindrical tube is configured to reversibly and repeatedly deform from a relaxed state to a stretched state in which the cylindrical tube contracts in a radial direction and expands in a longitudinal direction to secure the soft tissue within the cylindrical tube.

2. The sleeve for soft tissue repair of claim 1, wherein the cylindrical tube is formed of a plurality of strips of the at least one biocompatible material, and wherein the strips of the at least one biocompatible material are braided together.

3. The sleeve for soft tissue repair of claim 1, wherein the cylindrical tube is comprised of a mesh material formed by interlacing wires or fibers of the at least one biocompatible material.

4. The sleeve for soft tissue repair of claim 1, wherein the biocompatible material comprises a suture material, a tissue graft, a plastic, a polymer, or any combination thereof.

5. The sleeve for soft tissue repair of claim 1, wherein the cylindrical tube is formed of a plurality of biocompatible materials.

6. The sleeve for soft tissue repair of claim 1, wherein the cylindrical tube includes a biologic agent configured to assist with healing the soft tissue.

7. The sleeve for soft tissue repair of claim 6, wherein the biologic agent comprises a sugar, a protein, a nucleic acid, or a mixture of any two or more thereof.

8. The sleeve for soft tissue repair of claim 6, wherein the biologic agent comprises stem cells, growth factors, hormones, blood, tendons, ligaments, bone, or a mixture of any two or more thereof.

9. The sleeve for soft tissue repair of claim 1, wherein the suture comprises an asymmetric suture including a first region proximate to a first end of the asymmetric suture, a second region, and a third region proximate to a second end of the asymmetric suture, the second region being asymmetric with respect to the first region and the third region.

10. The sleeve for soft tissue repair of claim 1, wherein the suture is configured to pass through the cylindrical tube having the soft tissue secured therein to form a medial row extending between a top surface and a bottom surface of the soft tissue.

11. The sleeve for soft tissue repair of claim 1, wherein the suture is configured to form a lateral row across a top surface of the cylindrical tube in a direction extending from a first point on a top surface of the soft tissue to a second point on the top surface of the soft tissue.

12. The sleeve for soft tissue repair of claim 1, wherein the suture is configured to pass through the cylindrical tube having the soft tissue secured therein to form a medial row extending between a top surface and a bottom surface of the soft tissue, and wherein the suture is further configured to form a lateral row across a top surface of the cylindrical tube in a direction extending from a first point on the top surface of the soft tissue to a second point on the top surface of the soft tissue.

13. The sleeve for soft tissue repair of claim 1, wherein the biocompatible material is arranged such that the cylindrical tube is continuous along an entire length thereof.

14. A method for soft tissue repair, the method comprising:
providing a sleeve over a soft tissue to be repaired, the sleeve comprising a cylindrical tube formed of at least one biocompatible material, and a suture tethered to one end of the cylindrical tube;
pulling the suture to deform the cylindrical tube from a relaxed state to a stretched state in which the cylindrical tube contracts in a radial direction and expands in a longitudinal direction to secure the soft tissue within the cylindrical tube; and
securing the suture to maintain a radial force imparted to the soft tissue by the sleeve in the stretched state.

15. The method of claim 14, wherein the step of securing the suture comprises securing the suture to at least one suture anchor configured to secure the suture to the soft tissue, a muscle, or a bone.

16. The method of claim 14, further comprising passing the suture through the sleeve having the soft tissue secured therein to form a medial row extending between a top surface and a bottom surface of the soft tissue, prior to the step of securing the suture.

17. The method of claim 14, further comprising forming a lateral row across a top surface of the sleeve in a direction extending from a first point on a top surface of the soft tissue to a second point on the top surface of the soft tissue, prior to the step of securing the suture.

18. The method of claim 17, wherein the suture comprises an asymmetric suture including a first region proximate to a first end of the asymmetric suture, a second region, and a third region proximate to a second end of the asymmetric suture, the second region being asymmetric with respect to the first region and the third region, and wherein the second region of the asymmetric suture forms the lateral row.

19. The method of claim 14, wherein, prior to the step of securing the suture, the method further comprises:
passing the suture through the sleeve having the soft tissue secured therein to form a medial row extending between a top surface and a bottom surface of the soft tissue; and
forming a lateral row across a top surface of the sleeve in a direction extending from a first point on the top surface of the soft tissue to a second point on the top surface of the soft tissue, prior to the step of securing the suture.

20. The method of claim 14, further comprising a step of introducing a biologic agent into the sleeve to assist with healing the soft tissue, wherein the biologic agent comprises a sugar, a protein, a nucleic acid, a stem cell, a growth factor, a hormone, blood, a tendon, a ligament, bone, or a mixture of any two or more thereof.
